(19) United States
(12) Patent Application Publication
(10) Pub. No.: US 2002/0091337 A1
(43) Pub. Date: Jul. 11, 2002

(54) Wireless communications system for implantable hearing aid

(76) Inventors: Theodore P. Adams, Edina, MN (US); Kai Kroll, Minneapolis, MN (US); Bruce A. Brillhart, Minneapolis, MN (US)

Correspondence Address: Fredrikson & Byron, P.A., 1100 International Centre, 900 Second Avenue South, Minneapolis, MN, 55402, US

(21) Appl. No.: 09/778,821

(22) Filed: Feb. 7, 2001

Related U.S. Application Data
(60) Provisional application No. 60/180,509, filed on Feb. 7, 2000.

(51) Int. Cl. A61B 5/00; H04R 25/00

(52) U.S. Cl. 600/559; 600/25

(57) ABSTRACT

A wireless signal processing device integrated with a hearing device is structured to form a wireless auditory system. Specifically, the system enables reception of radio and similar related broadcast, within a selected range of frequencies, directly into the hearing aid. A remote programmable unit operates as a transceiver and a selector to provide the user with several options to change frequencies, adjust volume and select among broadcast programs. A receiver is integrated with the hearing device to wirelessly receive transmission signals from the programmer unit. The hearing device may be fitted with a transceiver unit to directly receive and transmit signals. In this arrangement, the programmer unit is used to control channel, frequency and volume while simultaneously serving as a redundant reception and transmission unit. The system could be used to enable direct transfer of information, communication and entertainment in addition to its normal function as a hearing aid unit.

### BACKGROUND OF THE INVENTION

1. Field of the Invention

The present invention relates generally to implantable hearing aid technology. Specifically, the invention pertains to a wireless transfer and management of information in an implantable hearing aid device such that communications from radio, TV sound, intercom, telecom, cellular systems, computer generated sound and similar audio stimulus may be remotely received and heard via the implantable hearing device.

2. Description of Related Art

In most types of partial middle ear implantable (P-MEI) or total middle ear implantable (T-MEI) hearing assistance systems, sounds produce mechanical vibrations which are transduced by an electromechanical input transducer into electrical signals. These electrical signals are in turn provided to an electronics unit which amplifies the signals to subsequently feed into an electromechanical output transducer. The electromechanical output transducer vibrates an ossicular bone in response to the applied amplified electrical signals. The vibration is communicated to the inner ear and, ultimately, hearing is improved.

Although these types of hearing devices, as well as other hearing assistance systems, typically involve various mechanisms and electronics to convert mechanical vibrations to audible sound, they are not equipped to directly receive electronic signals from radio, TV and similar broadcast audio transmissions. Specifically, patients who wear hearing aid devices do not have the option to directly select and adjust broadcast frequencies adapted to their particular hearing aid features. Accordingly there is a need for a hearing device that is compatible with a wireless system to enable reception of a selectable set of frequencies from radio, TV and similar broadcast. Further there is a need to receive wireless transmission directly into a hearing aid device without an intermediate audio amplification or modification system.

### SUMMARY OF THE INVENTION

Accordingly, this invention provides a built-in receiver to receive broadcast from an external transmitter to directly receive RF broadcast in a hearing aid. A built in radio system with, preferably, a transceiver may be implemented in a hearing device to enable direct tuning and reception of certain broadcast programs. More specifically, a direct communication pipeline of information and entertainment is advantageously integrated with a hearing aid to enable a patient to directly access RF and similar transmissions. Optionally, a microphone may be implemented in any embodiment of this invention as a fail safe unit to enable the hearing device to receive a transmission in the event of failure of reception elements.

Another embodiment of this invention provides a hand held transmitter that is adjustable and programmable to receive and transmit selected broadcast from radio, TV, cellular phone and any similar broadcasting device directly into the hearing aid device.

In a further embodiment, a partially implantable unit including an RF link and a microphone and or transmitter assembled and adapted for cranial, subcranial, pectoral and dorsal implantation or to be worn externally, for example, as a necklace is used with the hearing assistance system.

Accordingly the present invention generally and without limitations provides, inter alia, a broadcast receiver electronics built into the hearing device. The receiver electronics could implement a lead system from a transducer as an antenna. An external, preferably hand held, wireless programmer is used by the patient to control the hearing aid features including channel and frequency selections. Independent volume controls enable volume management and control from the broadcast unit and the input transducer. Alternately, the wireless programmer may be equipped to both receive and transmit signals to a receiver in the hearing aid.

### BRIEF DESCRIPTION OF THE DRAWINGS

FIG. 1 illustrates a section of an anatomically normal human ear in which the present invention is implemented.

FIG. 2A shows in block diagrams a representative circuit of a receiver.

FIG. 2B shows in block diagrams a representative circuit of a transmitter.

FIG. 2C shows in block diagrams a representative circuit of a transceiver.

FIG. 2D shows in block diagrams the implementation of the present invention, fitted in a human ear.

FIG. 3 shows an alternate embodiment of the integration of the present invention with a middle ear implant hearing device.

FIG. 4 shows an embodiment in which a microphone is integrated with an external transmitter unit.

FIG. 5 shows a high level logic of the programmer as implemented in the present invention.

FIG. 6 shows a high level logic of a transceiver device implemented in the programmer and a unit in the hearing device.

### DESCRIPTION OF THE INVENTION

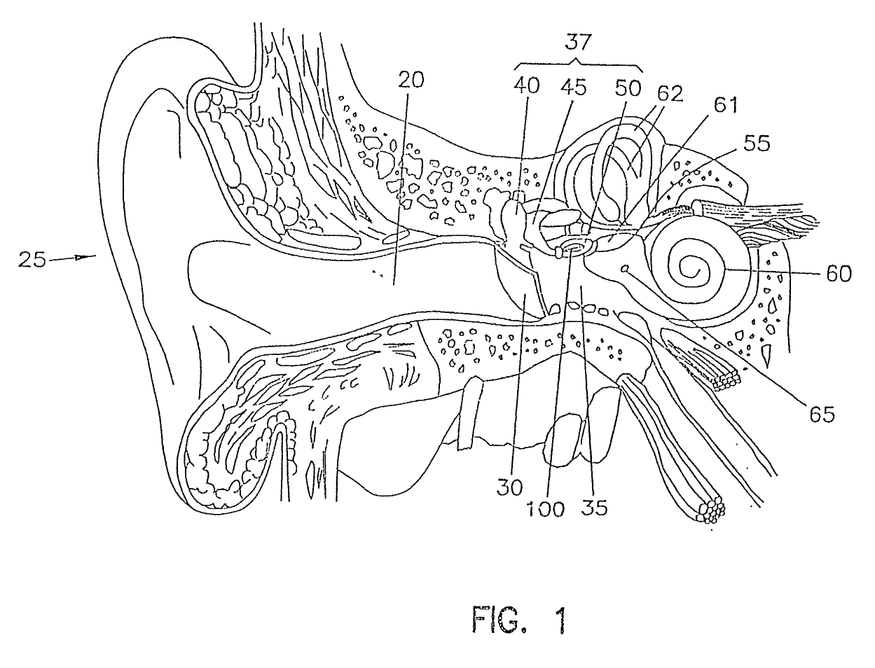

FIG. 1 illustrates generally the use of the invention in a human auditory system. Sound waves are directed into an external auditory canal 20 by an outer ear (pinna) 25. The frequency characteristics of the sound waves are slightly modified by the resonant characteristics of the external auditory canal 20. These sound waves impinge upon the tympanic membrane (eardrum) 30, interposed at the terminus of the external auditory canal 20, between it and the tympanic cavity (middle ear) 35. Variations in the sound waves produce tympanic vibrations. The mechanical energy of the tympanic vibrations is communicated to the inner ear, comprising cochlea 60, vestibule 61, and semicircular canals 62, by sequence of articulating bones located in the middle ear 35. This sequence of articulating bones is referred to generally as the, ossicular chain 37. Thus, the tympanic membrane 30 and ossicular chain 37 transform acoustic energy in the external auditory canal 20 to mechanical energy at the cochlea 60.

The ossicular chain 37 includes three primary components: a malleus 40, incus 45, and a stapes 50. The malleus 40 includes manubrium and head portions. The manubrium of the malleus 40 attaches to the tympanic membrane 30. The head of the malleus 40 articulates with one end of the incus 45. The incus 45 normally couples mechanical energy from the vibrating malleus 40 to the stapes 50. The stapes 50 includes a capitulum portion, comprising a head and a neck, connected to a footplate portion by means of a support crus comprising two crura. The stapes 50 is disposed in and against a membrane-covered opening on the cochlea 60. This membrane-covered opening between the cochlea 60 and middle ear 35 is referred to as the oval window 55. Oval window 55 is considered part of cochlea 60 in this patent application. The incus 45 articulates the capitulum of the stapes 50 to complete the mechanical transmission path.

Normally, prior to implantation of the invention, tympanic vibrations are mechanically conducted through the malleus 40, incus 45, and stapes 50, to the oval window 55. Vibrations at the oval window 55 are conducted into the fluid-filled cochlea 60. These mechanical vibrations generate fluidic motion, thereby transmitting hydraulic energy within the cochlea 60. Pressures generated in the cochlea 60 by fluidic motion are accommodated by a second membrane-covered opening on the cochlea 60. This second membrane-covered opening between the cochlea 60 and middle ear 35 is referred to as the round window 65. Round window 65 is generally considered part of cochlea 60 in this patent application. Receptor cells in the cochlea 60 translate the fluidic motion into neural impulses which are transmitted to the brain and perceived as sound. However, various disorders of the tympanic membrane 30, ossicular chain 37, and/or cochlea 60 can disrupt or impair normal hearing.

A piezoelectric output transducer is also capable of effecting mechanical vibrations to the ossicular chain 37. An example of such a device is disclosed in U.S. Pat. No. 4,729,366, issued to D. W. Schaefer on Mar. 8, 1988. In the '366 patent, a mechanical-to-electrical piezoelectric input transducer is associated with the malleus 40, transducing mechanical energy into an electrical signal, which is amplified and further processed. A resulting electrical signal is provided to an electrical-to-mechanical piezoelectric output transducer that generates a mechanical vibration coupled to an element of the ossicular chain 37 or to the oval window 55 or round window 65. In the '366 patent, the ossicular chain 37 is interrupted by removal of the incus 45. Removal of the incus 45 prevents the mechanical vibrations delivered by the piezoelectric output transducer from mechanically feeding back to the piezoelectric input transducer.

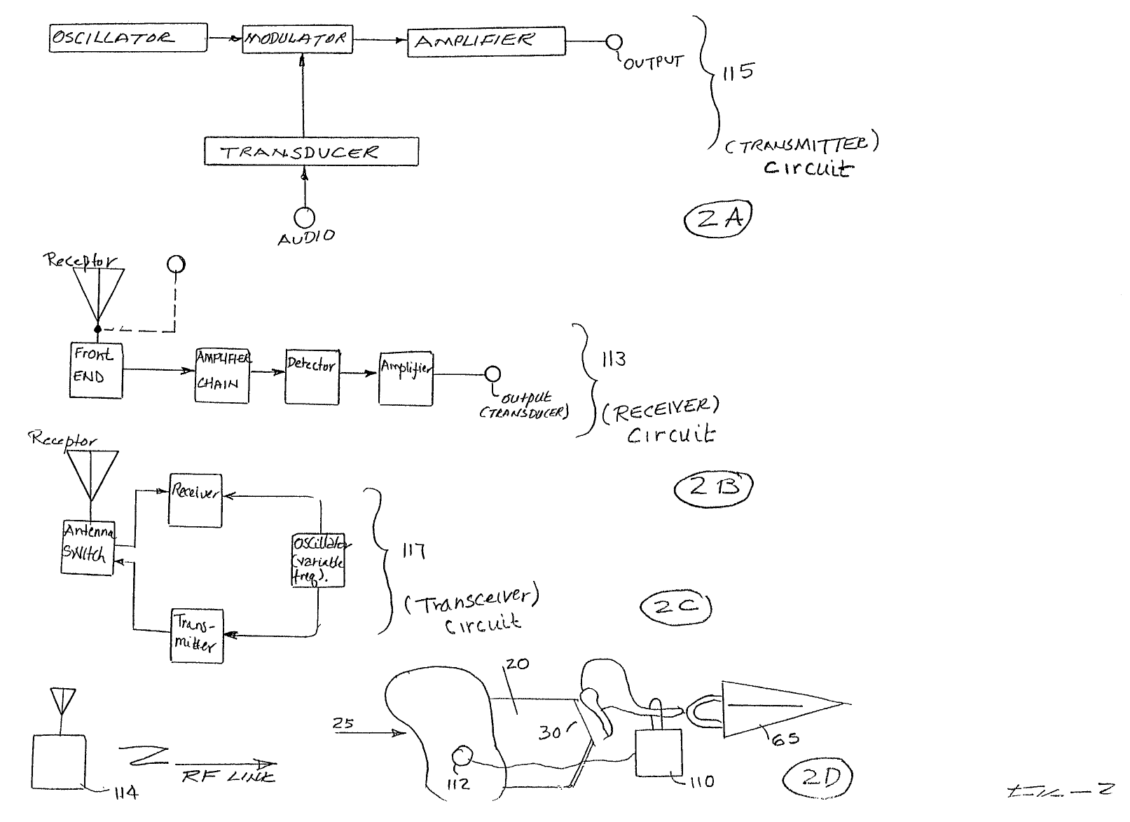

Referring now to FIGS. 2A-2D, an implantable middle ear hearing prosthesis includes a radio receiver 113 built into the electronics package. Generally, unit 112 is preferably structured in the manner of the receiver 113 wherein a receptor is wirelessly influenced by a radio or any similar frequency input. The input is fed into a front end or wide band filter which provides amplification and selectivity. The amplification chain increases the weak signal to a level sufficient to operate the detector which extracts the modulation information from the RF energy. The audio amplifier provides sufficient amplitude to the detached signal to drive transducer 110. Subsequently, transducer 110 converts the detected signal to a form suitable for listening. Further, transmitter 115 is preferably structured for producing a signal or signals for broadcasting or communications purposes. Optionally, the signal consists of an electric current, radio waves, light, ultrasound, or any other compatible form of energy. The transmitter converts audio information into a signal to be sent to unit 112. Transmitter 115 preferably includes an oscillator, a transducer, a modulator and a signal amplifier. The amplifier output is connected to an antenna system. The oscillator provides the carrier wave. The transducer converts audio information into electrical signals. The modulator impresses the output of the transducer onto the carrier wave. The amplifier increases the signal level to provide sufficient power for transmission over the required distance. The present invention may incorporate mixers to obtain mulitiband operation over the transmitter. In an alternate embodiment, audio information could be received by using the lead system to transducer 110 as an antenna.

FIG. 2C shows transceiver 117 depicted in block diagrams.

Programmer unit 114 includes transceiver 117 in addition to a receiver and a transmitter with a common frequency control. The principal components include a variable-frequency oscillator or channel synthesizer, a transmitter, a receiver and an antenna switching device. Generally, programmable unit 114 enables the user to select and set up frequencies and channels. Alternately, transceiver circuit 117 is advantageously incorporated into (receiver) unit 112. In this embodiment, programmer unit 114 is preferably used for channel and frequency selection, volume adjustment and related functions.

The present invention is intended to provide therapy to the broad patient population suffering from hearing loss ranges of 60 to 90 decibels. An implementation of the present invention may include the disarticulation or removal of one or more elements in the ossicular chain 37. Disarticulation enables detection of sound from within the middle ear without the use of a microphone and without the possibility of acoustic feedback.

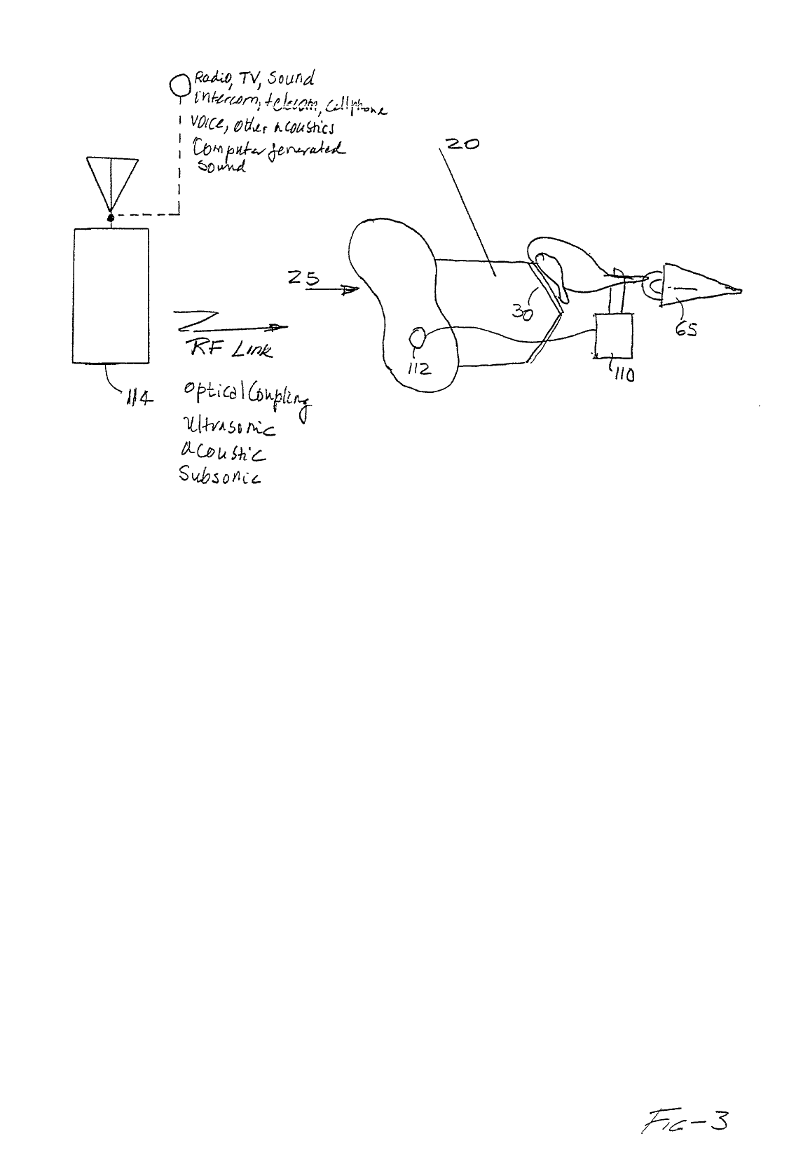

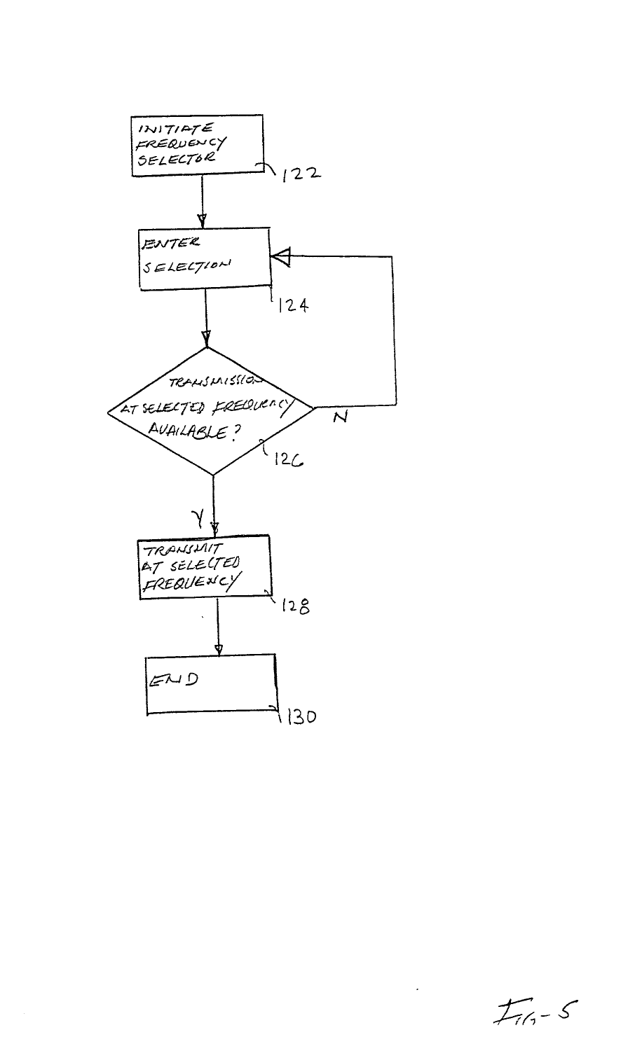

Referring now to FIG. 3 programmer 114 receives radio, TV, intercom, telecom, voice and similar signals. Programmer unit 114 may include transceiver 117 and could be programmed to transmit a selected set of signals, which selection is made by the user (see FIG. 5). The selected entry of frequencies are isolated by the programmer and wirelessly transmitted to receiver 112. The received impulse is introduced into transducer 110 and communicated to cochlea 65 wherein the auditory vibration is changed into nerve impulses for perception by the brain.

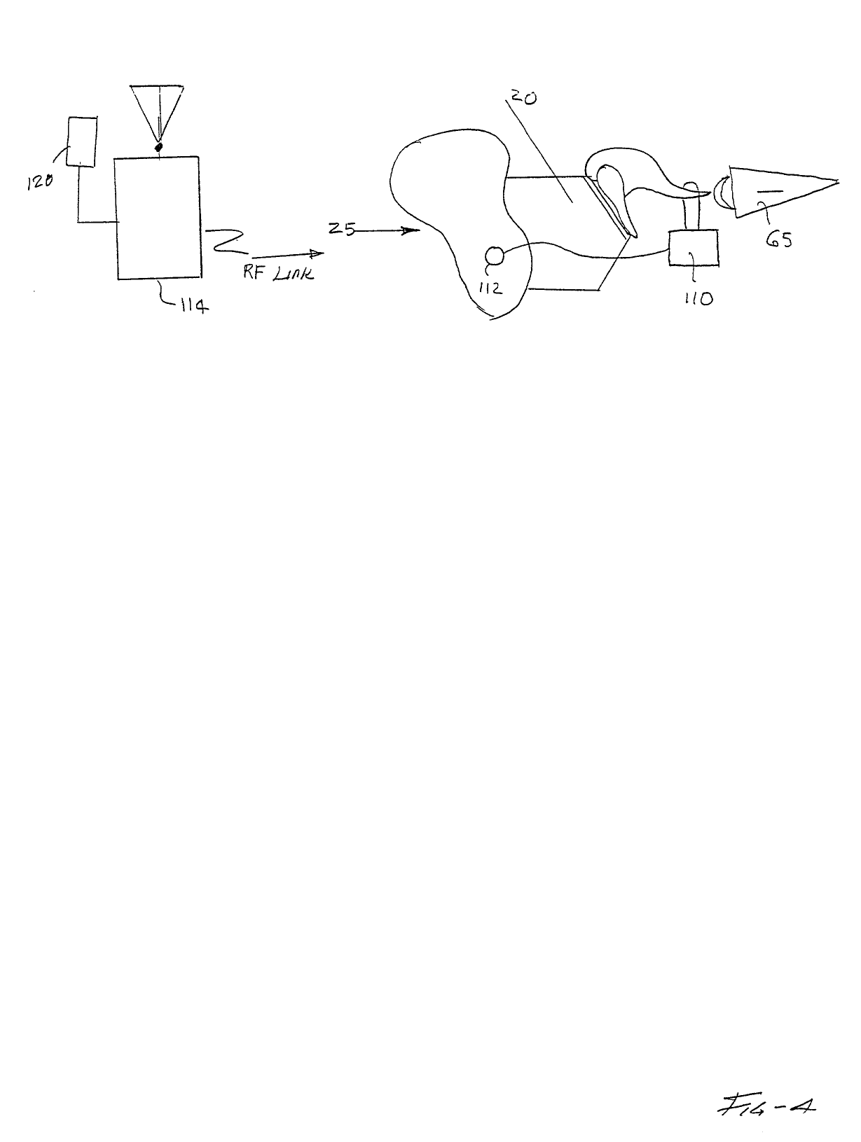

FIG. 4 shows the implementation of a fail safe device such as microphone 120. If programmer 114 fails, microphone 120 could be used to provide audible sound to transducer 110.

Referring now to FIG. 5, a high level operational program logic of the hand held transceiver/programmer unit 114 is shown. Specifically, the program is started by the user under logic step 122 wherein the frequency selector is initiated. The user is prompted under logic step 124 to enter the selection of frequencies for reception by the implanted (receiver) unit 112. Preferably, (receiver) unit 112 is set to receive specific frequencies from programmer 114. Once the selection is made under logic step 124, the program logic proceeds to decision block 126 where the system checks to see if there is transmission at the selected frequency. If the selected frequency does not match the available selection, the program logic reverts back to logic step 124 and prompts the user to enter another selection. Alternately, if the selected frequency is available, the program logic proceeds to logic step 128 where the transmission of the signal is executed at the selected frequency. Hereafter, the user may adjust the volume as needed and the session ends at logic step 130.

A further alternate embodiment includes the incorporation of transceiver circuit 117 in (receiver) unit 112. This embodiment enables direct reception of a wireless transmission in the outer ear for subsequent transfer to transducer 110 and eventual auditory perception. In this embodiment, programmer unit 114 is used to adjust reception volume and select frequencies. Programmer unit 114 further functions as a fail safe redundant system in the event of failure of (receiver) unit 112. Specifically, when unit 112 is fitted with circuit 117, a transceiver is used to directly and wirelessly receive auditory RF signals and programmer 114 is used as a wireless channel and frequency selection system. However, if the transceiver circuit in unit 112 is malfunctioning, the transceiver in programmer 114 may be used to receive, transmit and program the user's selections, thus acting as a backup system for unit 112.

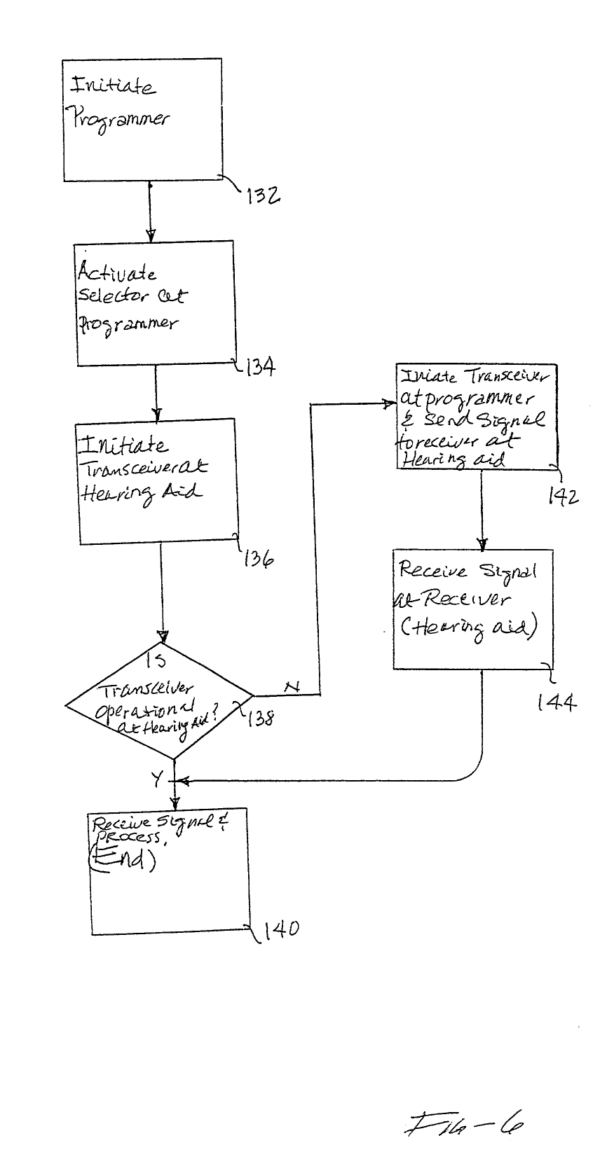

FIG. 6 shows a flow chart in which both programmer 114 and unit 112 are fitted with transceiver circuit 117. The program starts at logic step 132 by initiating programmer 114. Subsequently, a selector is activated at programmer 114 under logic step 134. Further, the transceiver circuit at unit 112 in the hearing aid is initiated under logic step 136. The subsequent logic includes decision block 138 in which the operational integrity of the transceiver in circuit 112 is verified. If the transceiver is functional, the program logic proceeds to logic step 140 where the signal is received in unit 112 and the selections and adjustments made as needed. In the alternate, if the transceiver in unit 112 is not operational, the program logic proceeds to logic step 142 where the transceiver at the programmer is initiated and the signal is sent to the unit 112 which is implemented to function as a receiver. Subsequently the signal is received by the receiver in unit 112 under logic step 144. Thereafter the program logic advances to logic step 140 where the signal is received by the hearing aid and processed for auditory perception.

The invention may alternately be used in the treatment of tinnitus. Tinnitus may be defined as “ringing” ears and similar head noises that are perceived without any external noise source or auditory stimulation. It is estimated that nearly 20% of the United States population experience a form of tinnitus. One method of treating tinnitus is by masking the sound. Masking involves the technique of generating external “white noise” sounds that mask the tinnitus to make it less audible to the patient and therefore less distracting. Masking devices come in both in-the-ear and portable models to produce sounds ranging from random white noise and other structured noise. Frequencies used are generally within a 1KHz-12KHz band. The RF signal of the present invention is advantageously adaptable to operate as a masker for tinnitus. Specifically, programmer unit 114 may be tuned to the proper AM/FM frequency to enable proper masking of the tinnitus. The hearing aid of the present invention enables fine tuning and adjustment of the RF signal to provide effective masking.

Although the description of the preferred embodiment has been presented, it is contemplated that various changes could be made without deviating from the spirit of the present invention. Accordingly, it is intended that the scope of the present invention be dictated by the appended claims, rather than by the description of the preferred embodiment.

What is claimed is:

1. A wireless system integrated with a hearing device to enable the device to receive radio and similar related broadcast within a selected range of frequencies, the wireless system comprising:
a hand held programmable device; and
a hearing device in wireless communication with the programmable device and configured to receive radio frequency and similar type broadcast, the hearing device further comprising a receiver to receive programmed transmissions within a selected range of frequencies from the programmable device.

2. The wireless system of claim 1, wherein the programmable device includes a transceiver circuit.

3. The wireless system of claim 1, wherein the receiver incorporates a transceiver to directly receive radio frequency broadcast.

5. The wireless system of claim 1, wherein the programmable device incorporates a microphone.

6. The wireless system of claim 1 wherein the hearing device further comprises:
a transmitter in communication with the receiver; and
an output transducer in communication with the transmitter.

7. The wireless system of claim 6, wherein the transmitter further comprises an oscillator, a modulator, and an amplifier.

8. In a hearing device, a wireless system integrated with the hearing device to enable direct reception and transmission of an auditory signal, the wireless system comprising:
a transceiver unit implanted in a patient's ear;
a programmer in wireless communication with the transceiver unit; and
a transducer in electronic communication with the transceiver unit and operatively coupled to an ossicular element of the middle ear;
wherein the transceiver unit is structured to receive signals and transmit the signals to the transducer based on one of channel and frequency selections entered into the programmer.

9. The system of claim 8 wherein the programmer incorporates a microphone.

10. The system of claim 8 wherein the transceiver unit includes a receiver for receiving one of radio, telephone, television and related broadcast signal.

11. The system of claim 10 wherein the broadcast signal is selectably adapted for reception by the transceiver.

12. The system of claim 11 wherein the broadcast signal is transmitted into a receiver and a transducer for auditory perception in the inner ear.

13. The system of claim 12 wherein one of the auditory elements in the ossicular chain is disarticulated and the broadcast signal is transmitted across a gap in the ossicular chain.

14. The system of claim 8 wherein the programmer comprises a receiver and a transmitter.

15. The system of claim 8, wherein the programmer further comprises a transceiver with a common frequency control.

16. The system of claim 8 wherein the programmer is programmed to transmit a selected set of signals.

17. The system of claim 8 wherein the programmer and the transceiver unit each include a transceiver.

18. The system of claim 8 wherein the transceiver unit further includes an oscillator, a modulator, and an amplifier.

19. The system of claim 8 further comprising a volume protection unit.

20. In a hearing device, a method of receiving and transmitting a broadcast signal into an inner ear wherein a programmer and a unit in the hearing device are in wireless communication, the device-implemented method comprising the steps of:
receiving the broadcast signal by a transceiver in one of the programmer and the unit in the hearing device;
selecting a broadcast channel and adjusting volume via the programmer;
receiving the channel and volume selection via a receiver in the unit in the hearing device; and
transmitting the signal to a transducer for output to the inner ear.

21. The method according to claim 20 wherein both the programmer and the unit in the hearing device are fitted with a transceiver and the broadcast signal is directly received by the unit in the hearing device.

22. The method according to claim 21 wherein the transceiver in the programmer is initiated as a-backup only when the transceiver in the unit fails to perform one of-reception and transmission functions.

23. The method according to claim 20 wherein the unit in the hearing aid receives the broadcast signal from the programmer unit.

24. The method of claim 20 wherein the unit in the hearing device transfers the broadcast signal across a disarticulated chain of ossicular elements to a transducer in the inner ear.

25. The method of claim 24 wherein the unit in the hearing device is in electronic communication with the transducer across a gap of the disarticulated chain of ossicular elements.

26. The method of claim 20 wherein the unit in the hearing aid device receives the broadcast signal directly and wirelessly from a broadcast source.

27. The method according to claim 26 wherein the unit in the hearing aid device is in wireless communication to programmably adjust volume, select channels and perform frequency selections.

28. The method of claim 30 wherein at least one component is implanted in the middle ear.

29. The method of claim 22 wherein substantially all components of the hearing device are implanted in the middle ear.

30. The method of claim 20 wherein one of a universal masker and a specified radio frequency channel is accessed via the programmer.

31. The method of claim 20 wherein a lead in a transducer of the hearing is used as an antenna for reception of a broadcast.

32. The method of claim 20 wherein a volume protection unit is implemented to eliminate noise levels beyond a specified threshold.
